(19) United States
(12) Patent Application Publication
(10) Pub. No.: US 2016/0038553 A1
(43) Pub. Date: Feb. 11, 2016

(54) NUTRITIONAL SUPPLEMENT COMPRISING HERBAL GALACTAGOGUE

(71) Applicants: Kelly Andrews, Bellingham, WA (US); Suzanne Munson, Bellingham, WA (US)

(72) Inventors: Kelly Andrews, Bellingham, WA (US); Suzanne Munson, Bellingham, WA (US)

(73) Assignee: FAIRHAVEN HEALTH, LLC, Bellingham, WA (US)

(21) Appl. No.: 14/453,527

(22) Filed: Aug. 6, 2014

(51) Int. Cl. A61K 36/48; A61K 36/235; A23L 1/303; A23L 1/304; A23L 1/30; A23L 1/302

(52) U.S. Cl. CPC A61K 36/48; A23L 1/3002; A23L 1/302; A23L 1/303; A23L 1/304; A61K 36/235; A23V 2002/00

(57) ABSTRACT

The present invention provides a single unit composition for lactation improvement comprising herbal galactagogue and a method for using the same. In one particular embodiment, the single unit composition of the invention comprises fenugreek and a mixture of essential minerals and vitamins.

### FIELD OF THE INVENTION

The present invention relates to a nutritional supplement for the nursing mother. The nutritional supplement of the invention aids in improving lactation and milk flow and also provides a wide variety of essential vitamins and minerals.

### BACKGROUND OF THE INVENTION

Breast milk is the ideal nutrition source for infants. It provides complete nutrition that has yet to be adequately replicated by formula or other food. In addition to nutritional benefits, breastfeeding is also associated with better health and developmental outcomes both during infancy and later in life. Breastfeeding confers substantial immunological benefits and is associated with lower incidence of asthma, allergies, sudden infant death syndrome (SIDS), ear infections, respiratory illnesses, and diarrhea. Being breastfeed as an infant is associated with higher IQs and reduced incidence of psychological, behavioral, and learning problems in childhood and later life.

However, a portion of new mothers cannot produce breast milk in adequate quantities. Given that there is no replacement on par with breast milk, it is desirable to have a way of increasing or inducing lactation for those desiring to breastfeed but are unable to provide or maintain adequate milk supply.

Due to the nutritional demands of producing breast milk, women have higher nutrition requirements while nursing. Therefore, at least for the duration of lactation, it is desirable to supplement the intake of essential nutrients.

### SUMMARY OF THE INVENTION

Some aspects of the present invention provide diet supplement compositions comprising fenugreek source or extracts and methods for using the same, in particular in aiding the production of breast milk.

In some embodiments, the composition comprises fenugreek or its source or extract. When fenugreek source or its extract is used in the composition, the composition comprises at least about 10%, typically at least 25%, and often at least 50% fenugreek. Alternatively, the composition comprises an effective dose of fenugreek of at least about 500 mg, typically at least 750 mg, often at least 1,000 mg, and most often at least 2000 mg. Still in other embodiments, the composition further comprises fennel seed and a number of essential nutrients. In other embodiments the effective dose of fenugreek per single unit ranges from about 750 mg to about 1500 mg. Typically, oral administration of the composition of the invention is about 3 single units at a time. In some instances, compositions of the invention are administered once a day. Compositions of the invention can be used to induce and increase lactation, and provide supplementation of daily nutritional requirements.

One particular aspect of the invention provides a single unit nutritional supplement for breastfeeding female to enhance breast milk supply and/or lactation, said nutritional supplement comprising a postnatal vitamin, an essential mineral, and an effective amount of an herbal galactagogue.

In some embodiments, said herbal galactagogue comprises fenugreek, fennel seed, or a combination thereof. In some instances, said nutritional supplement comprises at least 750 mg of effective fenugreek. In other instances, said nutritional supplement comprises at least 2,000 mg of effective fenugreek.

Still in other embodiments, said herbal galactagogue comprises fenugreek seed powder, fenugreek extract, or a combination thereof.

Yet in other embodiments, said single unit nutritional supplement comprises vitamin D, vitamin B₆, vitamin B₁₂, chromium, folic acid, or a combination thereof. In one particular embodiment, said single unit nutritional supplement comprises vitamin D, vitamin B₆, vitamin B₁₂, chromium, folic acid, fenugreek, fennel seed, and at least one of an additional ingredient selected from the group consisting of vitamin A, vitamin C, vitamin E, vitamin K, thiamin, riboflavin, niacin, biotin, pantothenic acid, calcium, iron, iodine, magnesium, zinc, selenium, copper, manganese, coriander, anise, and chamomile. In some cases, said single unit nutritional supplement comprises at least five of said additional ingredients. In other cases, said single unit nutritional supplement comprises at least ten of said additional ingredients. Still in other instances, said single unit nutritional supplement comprises at least fifteen of said additional ingredients.

Another aspect of the invention provides a single unit nutritional supplement for breastfeeding female to enhance breast milk supply and/or lactation, said nutritional supplement comprising at least five postnatal vitamins, at least five essential minerals, and at least 1500 mg of effective fenugreek.

In some embodiments, the single unit nutritional supplement further comprises fennel seed.

Still in other embodiments, the postnatal vitamin Comprises vitamin A, vitamin C, vitamin D, vitamin E, vitamin K, vitamin B₆, vitamin B₁₂, riboflavin, thiamin, niacin, folic acid, biotin, pantothenic acid, or a combination thereof.

Yet in other embodiments, the essential mineral comprises calcium, iron, iodine, magnesium, zinc, selenium, copper, manganese, chromium or a combination thereof.

In other embodiments, said fenugreek comprises fenugreek seed powder, fenugreek extract, or a combination thereof.

Yet another aspects of the invention provides a method for improving lactation and/or breast milk production in a breastfeeding female, said method comprising administering to a breastfeeding female an effective dose of a single unit nutritional supplement disclosed herein. In some embodiments, the effective dose comprises no more than three single units of said nutritional supplement disclosed herein.

### DETAILED DESCRIPTION OF THE INVENTION

Fenugreek, Trigonella foenum-graecum, is a plant that is a galactagogue, a substance that can be used to promote lactation. Unfortunately, all conventional nutritional supplements containing fenugreek do not include other essential vitamins and minerals that a lactating female needs, thereby requiring the female to take separate vitamins and essential minerals supplements. This separate nutritional supplement requirement is cumbersome, results in non-compliance, and/or often results in females taking only one or the other nutritional supplements. Fenugreek may be consumed or administered in a variety of methods. Fenugreek is safe to consume as food, both leafy part of the plant and its seeds may be consumed. The seeds may be consumed, in whole or in part, and fenugreek seeds are a part of a variety of culinary traditions. Fenugreek may also be administered as powdered seed and as concentrated extract. The Food and Drug Administration classifies fenugreek in supplement form as Generally Regarded As Safe, i.e., “GRAS”.

“Single unit” refers to a single physical unit of the composition, such as a single tablet, capsule, gel caps, or the like. A single administration of the composition may comprise one or more single units, typically one to three single units. In some embodiments, a single administration comprises two or three single units. The term “effective dose” refers to the equivalent amount of a substance, regardless of form or source, taking into consideration dilution and concentration. In particular, the term “effective dose” when referring to fenugreek means the equivalent amount of fenugreek seed powder that a composition provides. Thus, for example, the effective dose of fenugreek of 1 mg of a tenfold concentrated extract is 10mg of fenugreek, while 10 mg of a 1:1 mixture of fenugreek and another, non-fenugreek substance would have an effective dose of only 5 mg of fenugreek. A mixture of 10 mg of fenugreek seed powder and 1 mg of a 10× concentrated fenugreek extract would thus be an effective dose of 20 mg of fenugreek.

“Essential mineral” refers to a trace element or mineral required for normal biological function that cannot be synthesized by the body in that is not a vitamin. Essential minerals include, but are not limited to: calcium, iron, iodine, magnesium, zinc, selenium, copper, manganese, and chromium. “Vitamin” refers to a vital nutrient that is an organic compound which must be obtained in whole or in part from external sources. There are currently thirteen known vitamins: vitamin A, vitamin B1 (thiamine), vitamin B2 (riboflavin), vitamin B3 (niacin, niacinamide), vitamin B5 (pantothenic acid), vitamin B6 (pyridoxine), vitamin B7(biotin), vitamin B9 (folic acid), vitamin B 12 (cyanocobalamin, methylcobalamin), vitamin C (ascorbic acid), vitamin D (cholecalciferol (D3), ergocalciferol (D2)), vitamin E, and vitamin K. “Percentage Daily Value” or “% DV” refers to the percentage of the recommended daily intake of a particular substance in as defined by the Food and Drug Administration. The % DV used herein is based on the recommended intake for pregnant and lactating women.

One specific embodiment of the composition is a single unit comprising at least an effective dose of 750 mg fenugreek. In some instances, the single unit composition also includes one or more of the following: at least 8.3% DV vitamin A, at least 8.3% DV vitamin C, at least 58.3% DV vitamin D3, at least 8.3% DV vitamin E, at least 16.7% DV thiamin, at least 16.7% DV riboflavin, at least 16.7% DV niacin, at least 16.7% DV vitamin B6, at least 8.3% DV folic acid, at least 16.7% DV vitamin B12, at least 16.7% DV biotin, at least 16.7% DV Pantothenic Acid, at least 0.3% DV calcium, at least 8.3% DV iron, at least 8.3% DV Iodine, at least 0.3% DV magnesium, at least 8.3% DV zinc, at least 4.2% DV copper, or a combination of two or more, typically three or more and often five or more thereof. Still in other instances, the single unit can also include at least 0.2 mg manganese, at least 10 mcg chromium, at least 6.7 mcg vitamin K, at least 5.8 mcg selenium. Yet in other instances, the single unit can also include other plant-derived substances such as fennel seed, coriander seed, anise seed, chamomile flower, and/or vervain herb. Still further, the single unit can also include inactive ingredients such as cellulose, rice flour, magnesium stearate, silicon dioxide, or a combination thereof

In another specific embodiment, the single unit composition of the invention comprises an effective dose of at least 1000 mg of fenugreek. In some instances, the single unit composition can also include one or more of the following: at least 16.7% DV vitamin A, at least 16.7% DV vitamin C, at least 116.7% DV vitamin D3, at least 16.7% DV vitamin E, at least 33.3% DV thiamin, at least 33.3% DV riboflavin, at least 33.3% DV niacin, at least 33.3% DV vitamin B6, at least 16.7% DV folic acid, at least 33.3% DV vitamin B12, at least 33.3% DV biotin, at least 33.3% DV Pantothenic Acid, at least 0.5% DV calcium, at least 16.7% DV iron, at least 16.7% DV Iodine, at least 0.7% DV magnesium, at least 16.7% DV zinc, at least 8.3% DV copper, at least 0.3 mg manganese, at least 20 mcg chromium, at least 13.3 mcg vitamin K, and at least 11.7 mcg selenium.

Yet in another embodiment, the single unit composition of the invention comprises an effective dose of at least 1500 mg of fenugreek. The single unit dose composition can also include one or more of the following: at least 25.0% DV vitamin A, at least 25.0% DV vitamin C, at least 175.0% DV vitamin D3, at least 25.0% DV vitamin E, at least 50.0% DV thiamin, at least 50.0% DV riboflavin, at least 50.0% DV niacin, at least 50.0% DV vitamin B6, at least 25.0% DV folic acid, at least 50.0% DV vitamin B12, at least 50.0% DV biotin, at least 50.0% DV Pantothenic Acid, at least 0.8% DV calcium, at least 25.0% DV iron, at least 25.0% DV Iodine, at least 1.0% DV magnesium, at least 25.0% DV zinc, at least 12.5% DV copper, at least 0.5 mg manganese, at least 30 mcg chromium, at least 20 mcg vitamin K, and at least 17.5 mcg selenium.

Still in another embodiment, the single unit composition of the invention comprises an effective dose of at least 2000 mg of fenugreek. The single unit composition can also include one or more of the following: at least 33.3% DV vitamin A, at least 33.3% DV vitamin C, at least 233.3% DV vitamin D3, at least 33.3% DV vitamin E, at least 66.7% DV thiamin, at least 66.7% DV riboflavin, at least 66.7% DV niacin, at least 66.7% DV vitamin B6, at least 33.3% DV folic acid, at least 66.7% DV vitamin B12, at least 66.7% DV biotin, at least 66.7% DV Pantothenic Acid, at least 1.0% DV calcium, at least 33.3% DV iron, at least 33.3% DV Iodine, at least 1.3% DV magnesium, at least 33.3% DV zinc, at least 16.7% DV copper, at least 0.3 mg manganese, at least 0.7 mcg chromium, at least 26.7 mcg vitamin K, and at least 23.3 mcg selenium.

One particular aspect of the invention comprises a single unit nutritional supplement for breastfeeding female to enhance breast milk supply and/or lactation, said nutritional supplement comprising a postnatal vitamin, an essential mineral, and an effective amount of an herbal galactagogue. In some embodiments, said herbal galactagogue comprises fenugreek, fennel seed, or a combination thereof. In other embodiments, said nutritional supplement comprises at least 750 mg of effective fenugreek. Yet in other embodiments, said nutritional supplement comprises at least 2,000 mg of effective fenugreek. As stated herein, said herbal galactagogue comprises fenugreek seed powder, fenugreek extract, or a combination thereof. Still in other embodiments, said single unit nutritional supplement comprises vitamin D, vitamin B₆, vitamin B₁₂, chromium, folic acid, or a combination thereof. In other embodiments, said single unit nutritional supplement comprises vitamin D, vitamin B₆, vitamin B₁₂, chromium, folic acid, fenugreek, fennel seed, and at least one of an additional ingredient selected from the group consisting of vitamin A, vitamin C, vitamin E, vitamin K, thiamin, riboflavin, niacin, biotin, pantothenic acid, calcium, iron, iodine, magnesium, zinc, selenium, copper, manganese, coriander, anise, and chamomile. In some instances, said single unit nutritional supplement comprises at least five, typically at least ten, and often at least fifteen of said additional ingredients.

Another aspect of the invention provides a single unit nutritional supplement for breastfeeding female to enhance breast milk supply and/or lactation, said nutritional supplement comprising at least five postnatal vitamins, at least five essential minerals, and at least 1500 mg of effective fenugreek. In some embodiments, the single unit nutritional supplement further comprises fennel seed. Yet in other embodiments, said postnatal vitamin comprises vitamin A, vitamin C, vitamin D, vitamin E, vitamin K, vitamin B₆, vitamin B₁₂, riboflavin, thiamin, niacin, folic acid, biotin, pantothenic acid, or a combination thereof. Still in other embodiments, said essential mineral comprises calcium, iron, iodine, magnesium, zinc, selenium, copper, manganese, chromium or a combination thereof. In other embodiments, said fenugreek comprises fenugreek seed powder, fenugreek extract, or a combination thereof.

Still another aspect of the invention provides a method for improving lactation and/or breast milk production in a breastfeeding female. Such a method comprises administering to a breastfeeding female an effective dose of a single unit nutritional supplement disclosed herein. In some embodiments, said effective dose comprises no more than three single units of said nutritional supplement disclosed herein.

It should be appreciated that combinations of the various ingredients and/or amount described herein form other embodiments. For example, in one particularly embodiment the single unit composition comprises at least 2000 mg of effective dose of fenugreek, at least 16.7% DV vitamin A, at least 16.7% DV vitamin C, at least 116.7% DV vitamin D3, at least 16.7% DV vitamin E, at least 50.0% DV thiamin, at least 50.0% DV riboflavin, at least 50.0% DV niacin, at least 50.0% DV vitamin B6, at least 25.0% DV folic acid, at least 50.0% DV vitamin B12, at least 50.0% DV biotin, at least 50.0% DV Pantothenic Acid, at least 1.0% DV calcium, at least 33.3% DV iron, at least 33.3% DV Iodine, at least 1.3% DV magnesium, at least 33.3% DV zinc, at least 16.7% DV copper, at least 0.3 mg manganese, at least 0.7 mcg chromium, at least 26.7 mcg vitamin K, and at least 23.3 mcg selenium. In this manner, a variety of single unit compositions are embodied within the present invention.

Additional objects, advantages, and novel features of this invention will become apparent to those skilled in the art upon examination of the following examples thereof, which are not intended to be limiting. In the Examples, procedures that are constructively reduced to practice are described in the present tense, and procedures that have been carried out in the laboratory are set forth in the past tense.

### EXAMPLES

One possible manufacturing process of the invention is as follows: raw materials with a valid Certificate of Analysis are received in locked quarantine cages. Containers are inspected for defects. Non-defective container contents are sampled in a clean environment and then tested in a laboratory setting for both physical characteristics of the material such as identity, potency, density, moisture, and contaminants such as heavy metals and microbes. Raw materials falling inspection are rejected. Raw materials passing all inspections are moved into inventory for use in production. Following proper procedure including making all required records, raw materials are weighed out and verified by an additional party for each batch. Batches are mixed according to instructions and verified for weight after mixing. A sample of the mixed batch is then processed according to the final form of the material. Resultant capsule or tablet is tested for various properties, such as disintegration, hardness, weight variance, potency, density, and friability to ensure quality. Products failing any tests are quarantined. If test capsule or tablet passes all inspections, the remainder of the batch is processed. Capsules are then polished. Tablets are then coated. Coated tablets are tested for hardness, friability, disintegration, weight variance, and thickness. Coated tablets that pass such tests and polished capsules are then inspected for flaws, run through a metal detector, and are tested for microbial contamination and potency. Once all inspections are passed, tablets and capsules are packaged. A sample of the packaged product is retained. The remainder of the product from the batch is inspected for quality and then released.

The foregoing discussion of the invention has been presented for purposes of illustration and description. The foregoing is not intended to limit the invention to the form or forms disclosed herein. Although the description of the invention has included description of one or more embodiments and certain variations and modifications, other variations and modifications are within the scope of the invention, e.g., as may be within the skill and knowledge of those in the art, after understanding the present disclosure. It is intended to obtain rights which include alternative embodiments to the extent permitted, including alternate, interchangeable and/or equivalent structures, functions, ranges or steps to those claimed, whether or not such alternate, interchangeable and/or equivalent structures, functions, ranges or steps are disclosed herein, and without intending to publicly dedicate any patentable subject matter. All references cited herein are incorporated by reference in their entirety.

1. A single unit nutritional supplement for breastfeeding female to enhance breast milk supply and/or lactation, said nutritional supplement comprising a postnatal vitamin, an essential mineral, and an effective amount of an herbal galactagogue.

2. The single unit nutritional supplement of claim 1, wherein said herbal galactagogue comprises fenugreek, fennel seed, or a combination thereof.

3. The single unit nutritional supplement of claim 2, wherein said nutritional supplement comprises at least 750 mg of effective fenugreek.

4. The single unit nutritional supplement of claim 3, wherein said nutritional supplement comprises at least 2,000 mg of effective fenugreek.

5. The single unit nutritional supplement of claim 1, wherein said herbal galactagogue comprises fenugreek seed powder, fenugreek extract, or a combination thereof.

6. The single unit nutritional supplement of claim 1, wherein said single unit nutritional supplement comprises vitamin D, vitamin B₆, vitamin B₁₂, chromium, folic acid, or a combination thereof.

7. The single unit nutritional supplement of claim 6, wherein said single unit nutritional supplement comprises vitamin D, vitamin B₆, vitamin B₁₂, chromium, folic acid, fenugreek, fennel seed, and at least one of an additional ingredient selected from the group consisting of vitamin A, vitamin C, vitamin E, vitamin K, thiamin, riboflavin, niacin, biotin, pantothenic acid, calcium, iron, iodine, magnesium, zinc, selenium, copper, manganese, coriander, anise, and chamomile.

8. The single unit nutritional supplement of claim 7, wherein said single unit nutritional supplement comprises at least five of said additional ingredients.

9. The single unit nutritional supplement of claim 7, wherein said single unit nutritional supplement comprises at least ten of said additional ingredients.

10. The single unit nutritional supplement of claim 7, wherein said single unit nutritional supplement comprises at least fifteen of said additional ingredients.

11. A single unit nutritional supplement for breastfeeding female to enhance breast milk supply and/or lactation, said nutritional supplement comprising at least five postnatal vitamins, at least five essential minerals, and at least 1500 mg of effective fenugreek.

12. The single unit nutritional supplement of claim 11 further comprising fennel seed.

13. The single unit nutritional supplement of claim 11, wherein said postnatal vitamin comprises vitamin A, vitamin C, vitamin D, vitamin E, vitamin K, vitamin B₆, vitamin B₁₂, riboflavin, thiamin, niacin, folic acid, biotin, pantothenic acid, or a combination thereof.

14. The single unit nutritional supplement of claim 11, wherein said essential mineral comprises calcium, iron, iodine, magnesium, zinc, selenium, copper, manganese, chromium or a combination thereof.

15. The single unit nutritional supplement of claim 11, wherein said fenugreek comprises fenugreek seed powder, fenugreek extract, or a combination thereof.

16. A method for improving lactation and/or breast milk production in a breastfeeding female, said method comprising administering to a breastfeeding female an effective dose of a single unit nutritional supplement of claim 1.

17. The method of claim 16, wherein said effective dose comprises no more than three single units of said nutritional supplement of claim 1.
